(19) United States
(12) Patent Application Publication
(10) Pub. No.: US 2014/0364720 A1
(43) Pub. Date: Dec. 11, 2014

(54) SYSTEMS AND METHODS FOR INTERACTIVE MAGNETIC RESONANCE IMAGING

(71) Applicant: General Electric Company, Schenectady, NY (US)

(72) Inventors: Robert David Darrow, Scotia, NY (US); Vivek Prabhakar Vaidya, Bangalore (IN)

(21) Appl. No.: 13/913,846

(22) Filed: Jun. 10, 2013

(51) Int. Cl. A61B 5/00; A61B 5/055

(52) U.S. Cl. CPC A61B 5/748; A61B 5/055; A61B 5/0037; A61B 5/743; A61B 5/749
USPC 600/410

(57) ABSTRACT

Embodiments of a method, a system, and a non-transitory computer readable medium for use in interactive magnetic resonance imaging are presented. An initial region of interest of a subject is scanned to acquire imaging data using an initial imaging protocol. Anatomical labeling information corresponding to a plurality of regions corresponding to the subject may be determined based on the acquired data and/or previously available information. Particularly, determining the anatomical labeling information may include identifying one or more features of interest corresponding to the initial region of interest. Further, input from an operator corresponding to a desired imaging task may be received interactively. The imaging protocol may be updated in real-time by selectively configuring one or more imaging parameters that optimize implementation of the desired imaging task based on the determined anatomical labeling information. A subsequent scan may be performed using the updated imaging protocol for completing the desired imaging task.

### BACKGROUND

Clinical diagnosis and treatment often rely on image-derived parameters corresponding to a region of interest (ROI) of a patient for managing a plurality of life-threatening medical conditions. Accordingly, various medical procedures employ imaging modalities such as X-ray fluoroscopy, computed tomography (CT), and magnetic resonance imaging (MRI) for generating high-fidelity images that aid in diagnoses and/or guidance of surgical or interventional devices in real-time. Typically, these imaging modalities employ specific scan configurations that allow acquisition and reconstruction of imaging data from a desired ROI of the patient.

X-ray and CT images, however, visualize soft tissues poorly, and thus, may not provide reliable images for use in assessing certain pathological conditions of the patient. Moreover, X-ray and CT imaging exposes the patient and an operator to ionizing radiation that may increase with procedural complexity. Accordingly, certain medical procedures employ MRI for enhanced characterization of soft tissues, bone marrow, brain, spine, blood flow, and tissue contrasts without use of the ionizing radiation typically used by X-ray and CT systems. Additionally, MRI may also allow for accurate localization of an interventional device, which in turn, allows accurate navigation of the device within the patient's vasculature without injuring surrounding tissues.

Conventionally, MRI systems follow a prescribe-ahead imaging model, where a technician pre-selects one or more scanning protocols based on a desired imaging task for use in generating images of the ROI of the patient. A medical practitioner analyzes these images for identifying anomalies in the ROI of the patient and prescribing appropriate treatment. Alternatively, the medical practitioner may request for further scans for determining additional information that may aid in an accurate diagnosis of the patient.

Typically, the further scans may entail prescription of different scanning protocols that modify scanning parameters suitable for scanning a desired portion of patient anatomy at a desired resolution and/or spatial coverage. For example, a medical practitioner may request for a further scan that focuses on a portion of an originally scanned ROI in a higher spatial resolution. Alternatively, the medical practitioner may request for scanning of a region surrounding the ROI to identify any complications such as bleeding that need to be corrected and/or to identify and label an anatomical landmark. Particularly, when performing a minimally-invasive interventional procedure, the medical practitioner may request for a focused scan for imaging a site of intervention, identification of anatomical features of interest, or guidance for device navigation.

Insertion as well as navigation of the interventional device within different branches of a vascular system, however, is a challenging procedure. Further, identification of the desired anatomical features of interest and/or an accurate imaging plane may be confounded due to noise and poor contrast between target structures and surrounding tissues. Additionally, dynamic prescription of scanning protocols and accurate selection of scanning parameters suitable for a specified imaging task are time consuming and often dependent on the operator's experience and skill.

Particularly, in absence of relevant experience, navigation to a desired region, selection of appropriate scanning protocols and/or landmark recognition from a novice operator may often result in inconsistent and/or inaccurate imaging. Such inaccurate estimations or inadequate coverage of clinically relevant parameters such as a location of a lesion derived from images reconstructed using erroneous configurations may lead to incorrect diagnosis, which in turn, may adversely affect patient health. Additionally, inadequate coverage of the desired ROI may necessitate additional data acquisition, which further increases exam duration, while also adding to patient discomfort. Moreover, even when experienced operators are available, manual intervention based imaging and navigation may become a bottleneck for large-scale deployment of image analysis techniques.

### BRIEF DESCRIPTION

According to certain aspects of the present disclosure, a magnetic resonance imaging system, a non-transitory computer readable medium, and a method for magnetic resonance imaging are disclosed. The method entails scanning an initial region of interest of a subject to acquire imaging data using an initial imaging protocol. Anatomical labeling information corresponding to a plurality of regions corresponding to the subject may then be determined based on the acquired data, previously available information, or a combination thereof. To that end, determining anatomical labeling information may include identifying one or more features of interest corresponding to the initial region of interest. Further, input from an operator corresponding to a desired imaging task may be received interactively. Subsequently, the imaging protocol may be updated in real-time by selectively configuring one or more imaging parameters that optimize implementation of the desired imaging task based on the determined anatomical labeling information. A subsequent scan may then be performed using the updated imaging protocol for completing the desired imaging task.

### DRAWINGS

These and other features, and aspects of embodiments of the present disclosure will become better understood when the following detailed description is read with reference to the accompanying drawings in which like characters represent like parts throughout the drawings, wherein:

FIG. 1 is a schematic representation of an exemplary MRI system for use in interactive imaging, in accordance with aspects of the present disclosure; and

FIG. 2 is a flowchart depicting an exemplary method for interactive MRI, in accordance with aspects of present disclosure.

### DETAILED DESCRIPTION

The following description presents exemplary systems and methods for interactive navigation of a human body during diagnostic imaging. Particularly, embodiments illustrated hereinafter disclose an anatomy-aware MRI system that may be configured to allow interactive and real-time navigation of the human body by even inexperienced operators. Embodiments of the anatomy-aware MRI system may also be configured to perform a variety of imaging tasks, such as imaging a target VOI relative to a current field of view (FOV), labeling features of interest during imaging, and tracking an interventional device based on interactive input received from an operator in real-time.

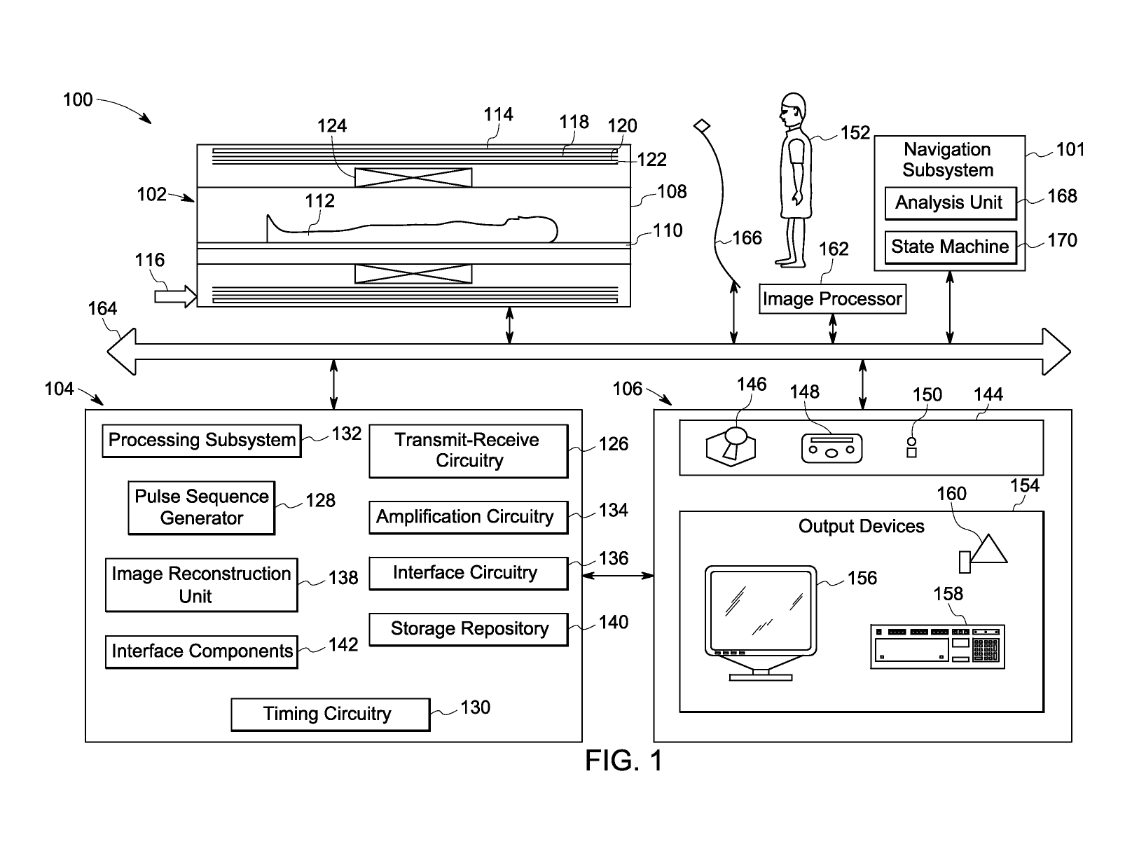

Although exemplary embodiments of the present systems and methods are described in the context of magnetic resonance (MR) imaging, it will be appreciated that use of embodiments of the present systems and methods in various other imaging applications and systems is also contemplated. For example, embodiments of the present systems and methods may be implemented in diagnostic, electrophysiological, surgical, and/or minimally-invasive systems such as intravascular ultrasound systems. Further, at least some of these systems and applications may also be used in non-destructive testing, fluid flow monitoring, and/or other chemical and biological applications. An exemplary environment that is suitable for practicing various implementations of the present system is discussed in the following sections with reference to FIG. 1.

FIG. 1 illustrates an MRI system 100 configured for interactive navigation of a human body during diagnostic, surgical, and/or interventional procedures. To that end, in one embodiment, the MRI system 100 includes a navigation subsystem 101, a scanner 102, a system controller 104, and an operator interface 106. In accordance with aspects of the present disclosure, the navigation subsystem 101 is configured to build comprehensive awareness of patient anatomy. To that end, the navigation subsystem 101, for example, may use projection data acquired using other system components such as the scanner 102. Additionally, the navigation subsystem 101 may also employ a surgical atlas, anatomical models, patient information gathered from previous examinations, system geometry, recommended scanning protocols, or other standardized information that may aid in identifying and labeling features of interest in a desired region.

The comprehensive anatomy awareness allows identification and labeling of features of interest at any coordinate in resulting images for use in providing real-time navigation guidance to even novice operators. Typically, the novice operators may not be skilled at, for example, configuring an optimized field of view (FOV) for imaging a particular region, configuring appropriate system parameters, or prescribing an optimal route for surgical and/or minimally-invasive intervention at a particular site. The anatomy aware navigation subsystem 101 may provide audio and/or visual indications or instructions to such novice users to achieve the optimal FOV or scan plane, system parameters suited to a specific imaging task and/or a suitable route for navigation of an interventional device through MR images generated in real-time. The MR images may be generated by scanning a desired volume of interest (VOI) of a patient 112.

To that end, in certain embodiments, the scanner 102 includes a patient bore 108 into which a table 110 may be positioned for disposing the patient 112 in a desired position for scanning. Moreover, the scanner 102 may also include a series of associated coils for imaging the patient 112. Particularly, in one embodiment, the scanner 102 includes a primary magnet coil 114, for example, energized via a power supply 116 for generating a primary magnetic field generally aligned with the patient bore 108. The scanner 102 may further include a series of gradient coils 118, 120 and 122 grouped in a coil assembly for generating accurately controlled magnetic fields, the strength of which may vary over a designated FOV of the scanner 102. In certain embodiments, the gradient coils 118, 120 and 122 may have different physical configurations adapted for different functions in the MRI system 100, such as, slice selection, frequency encoding, and/or phase encoding during MR imaging.

Further, the scanner 102 may include a radiofrequency (RF) coil 124 for generating RF pulses for exciting a gyromagnetic material, typically bound in tissues of the patient 112. In certain embodiments, the RF coil 124 may also serve as a receiving coil. Accordingly, the RF coil 124 may be operationally coupled to transmit-receive circuitry 126 in passive and active modes for receiving emissions from the gyromagnetic material and for applying RF excitation pulses, respectively. Alternatively, the MRI system 100 may include various configurations of receiving coils different from the RF coil 124. Such receiving coils may include structures specifically adapted for target anatomies, such as head, knee, and/or chest coil assemblies. Moreover, receiving coils may be provided in any suitable physical configuration, such as including phased array coils.

In certain embodiments, the system controller 104 controls operation of the associated MR coils for generating desired magnetic field and RF pulses. To that end, in one embodiment, the system controller 104 includes a pulse sequence generator 128, timing circuitry 130, and a processing subsystem 132 for generating and controlling imaging gradient waveforms and RF pulse sequences employed during a medical procedure. In one embodiment, the system controller 104 may also include amplification circuitry 134 and interface circuitry 136 for controlling and interfacing between the pulse sequence generator 128 and the coils of scanner 102. The amplification circuitry 134 may include one or more amplifiers that process the imaging gradient waveforms for supplying desired drive current to each of the gradient coils 118, 120, and 122 in response to control signals received from the processing subsystem 132. In certain embodiments, the amplification circuitry 134 may also amplify and couple the generated RF pulses to the RF coil 124 for transmission.

In one embodiment, the RF coil 124 receives response signals emitted by excited nuclei in the tissues of the patient 112. To that end, the RF coil 124 may be tuned to an imaging resonant frequency of the patient nuclei, for example, to about 63.5 MHz for hydrogen in a 1.5 Tesla magnetic field. In such embodiments, where the RF coil 124 serves both to emit the RF excitation pulses and to receive MR response signals, the interface circuitry 136 may also include a switching device (not shown in FIG. 1) for toggling the RF coil 124 between active/transmitting mode and passive/receiving mode. Additionally, the interface circuitry 136 may include additional amplification circuitry for driving the RF coil 124 and for amplifying the response signals for further processing. In certain embodiments, the amplified response signals may be transmitted to the processing subsystem 132 for determining information for use in image reconstruction.

Accordingly, the processing subsystem 132, for example, may include one or more application-specific processors, graphical processing units (GPUs), digital signal processors (DSPs), microcomputers, microcontrollers, Application Specific Integrated Circuits (ASICs) and/or Field Programmable Gate Arrays (FPGAs). In one embodiment, the processing subsystem 132 is configured to use interactive input received from an operator for reconfiguring a specific imaging protocol, for example, by customizing scan sequences and/or generating data indicative of the timing, strength, and shape of the RF and gradient pulses produced. Additionally, the processing subsystem 132 may ascertain the timing and length of a data acquisition window in the imaging pulse sequence using the timing circuitry 130.

In certain embodiments, the processing subsystem 132 processes the response signals emitted by excited patient nuclei in response to the RF pulses for providing navigational guidance and/or other diagnostic information. Specifically, the processing subsystem 132 may demodulate, filter, and/or digitize the response signals for determining the image reconstruction information. To that end, the processing subsystem 132 may be configured to apply analytical routines to the processed information for deriving features of interest, such as location of a stenosis and structural and/or functional parameters such as blood flow in the target VOI. The processing subsystem 132 may be configured to transmit this information to an image reconstruction unit 138 to allow reconstruction of desired images of the target VOI. Additionally, the processing subsystem 132 may be configured to receive and process patient data from a plurality of sensors (not shown in FIG. 1), such as electrocardiogram (ECG) signals from electrodes attached to the patient 112 for display and/or storage.

Accordingly, in certain embodiments, the system controller 104 may further include a storage repository 140 for storing the acquired data, reconstructed images, and/or related information derived therefrom. The storage repository 140 may also store physical and logical axis configuration parameters, pulse sequence descriptions, and/or programming routines for use during the scanning sequences implemented by the scanner 102. Additionally, the storage repository 140 may store the surgical atlas, anatomical models, patient information gathered from previous examinations, system geometry, recommended scanning protocols, or other standardized information that may aid in identifying and labeling features of interest during interactive MR imaging. Moreover, the storage repository 140 may also store comprehensive anatomical labeling information that may be determined from the acquired data or previously available information such as from the atlas or the previous examinations. To that end, the storage repository 140 may include devices such as a hard disk drive, a floppy disk drive, a compact disk-read/write (CD-R/W) drive, a Digital Versatile Disc (DVD) drive, a flash drive, and/or a solid-state storage device that may be configured to store both operation information as well as the comprehensive anatomical labeling information.

As previously noted, access to the anatomical labeling information corresponding to the patient 112 provides the navigation subsystem 101 with comprehensive awareness of the patient anatomy, specifically features of interest at any coordinate in resulting images. Such comprehensive anatomy awareness of every image coordinate in a VOI provides the MRI system 100 with ample flexibility to implement a variety of imaging tasks that are still unavailable in conventional MRI systems. For example, the MRI system 100 may provide a navigation route to the operator on the fly for imaging of a target region relative to a current view or switch between views in response to real-time vocal or touch-based input received via the operator interface 106 and interface components 142.

To that end, in one embodiment, the navigation subsystem 101 may retrieve the stored information such as scanning parameters, image data, and patient information from previous exams based on input received via the operator interface 106 and interface components 142. Particularly, in certain embodiments, the operator interface 106 may allow interactive selection of a desired imaging task, scanning parameters or selective patient information prior to, during, or after imaging. Accordingly, the operator interface 106 may further include one or more input devices 144 such as a keyboard, a mouse, a trackball, a joystick 146, a touch-activated screen, a light wand, a control panel 148, and an audio input device 150 such as a microphone associated with corresponding speech recognition circuitry for interactive imaging control and geometry prescription.

In one embodiment, the operator interface 106 allows for reconfiguration of imaging protocols and/or imaging parameters in real-time based on changes in imaging requirements. These requirements may be received from an operator, such as a medical practitioner 152 via input devices 144, or may be determined based on stored information corresponding to a medical procedure being undertaken. Specifically, in one embodiment, these requirements may include selection of a particular medical procedure, start and end points for imaging or intervention, or custom settings for one or more image parameters that are particularly suited to the imaging task at hand. Further, the imaging parameters, for example, may include parameters corresponding to flip angle, repetition time, FOV, spatial resolution, coverage, table and/or patient position, scanning time, scan sequence, shape and timing of the RF pulse sequences, or other such parameters specifically suited to the desired imaging task.

Further, in certain embodiments, the operator interface 106 may also include output devices 154 such as a display 156 including one or more monitors, printers 158 and/or an audio output device 160 such as speakers. The display 156, for example, may be integrated into wearable eyeglasses, or may be ceiling or cart mounted to allow the medical practitioner 152 to observe the reconstructed images, data derived from the images and other relevant information such as scanning time throughout the procedure. In one embodiment, the display 156 includes an interactive graphical user interface (GUI) that may allow selection and display of scanning modes, FOV, scan plane, navigation route, and prior exam data. The interactive GUI on the display 156 may also allow on-the-fly access to patient data such as respiration and heart rate, scanning parameters and selection of an ROI for subsequent imaging.

In one embodiment, the MRI system 100 may include a dedicated image processor 162, communicatively coupled to the navigation subsystem 101, for allowing real-time interactive MRI and navigation of the patient anatomy. To that end, the image processor 162 may be configured to receive the input parameters from the operator interface 106 via a communications link 164, such as a backplane or Internet, and process the received information to allow control over generation and display of MR images and/or image derived information before, during, or after imaging. Although the present disclosure describes the use of a dedicated image processor 162, in certain embodiments, the image processor 162 may be absent and one or more functions of the image processor 162 may be implemented by the navigation subsystem 101, processing subsystem 132 or the system controller 104, for example, by allowing all processes to run using a shared memory technique.

In a presently contemplated embodiment, the MRI system 100 employs the dedicated image processor 162 for reconstructing high quality images in real-time for use during a minimally-invasive interventional procedure. Specifically, the image processor 162 processes the received and/or stored image data to reconstruct and analyze the images. The image analysis aids in determining structural and/or functional information of the target VOI for diagnosis and/or treatment. Structural information such as location and size of a stenosis and functional information such as tissue perfusion parameters are very useful in ascertaining the pathological condition of the target VOI. The medical practitioner 152 may rely on these functional parameters before the surgical procedure to plan for appropriate therapeutic measures to be applied during endovascular treatments such as cerebral vascular accidents and angioplasties. The medical practitioner 152 may also use the functional parameters during the interventional procedure for evaluating effect of therapy in near real-time, and further for determining whether to stop or continue the procedure based on the evaluated effect.

Accordingly, in one embodiment, the medical practitioner 152 employs the MRI system 100 to provide information and/or high-fidelity images for performing an interventional procedure on the patient 112. During the interventional procedure, the medical practitioner 152 may insert a minimally-invasive interventional device 166 into an access site such as a vascular structure that provides access to the target VOI of the patient 112. In one embodiment, the access site may be in close proximity to the target VOI. In an alternative embodiment, however, the access site may be at a distance from the target VOI such that there is little or no overlap between an imaging FOV of the scanner 102 corresponding to the target VOI and the access site.

In certain embodiments, the movement of the interventional device 166 from the access site to the target VOI may be independently tracked by the navigation subsystem 101. The navigation subsystem 101, for example, may employ a reflective marker and/or a tracking sample that provides an MR signal at a resonant frequency, different from the resonant frequency of spins of protons used for imaging, for tracking the interventional device. Accordingly, the navigation subsystem 101 may be operationally coupled to the image processor 162 and/or the image reconstruction unit 138 to allow generation of MR images indicative of the movement of the interventional device 166 within the patient's body. Alternatively, in certain embodiments, the navigation subsystem 101 may directly determine a location of the interventional device 166 based on a position of an RF tracking coil or coils embedded in the interventional device 166.

Generally, for real-time hand-eye coordination, it is desirable that the time starting from the instant at which the interventional device 166 is moved to the time the acquired and processed data is displayed is less than 33 milliseconds (ms). Accordingly, in one embodiment, use of the dedicated image processor 162 allows for a separate image processing chain that aids in generating, for example, low-latency MRI images and corresponding diagnostic information. The low-latency MRI images and/or information, in turn, may be advantageously used for real-time guidance of the interventional device 166, thus providing functionality typically not available with conventional MRI systems.

In certain embodiments, the navigation subsystem 101 may configure the image processor 162 to register the MRI images to one or more predetermined images or a model of the target region such that position of the interventional device 166 may be tracked accurately on the display 156 with respect to surrounding regions. Particularly, in one embodiment, the MRI images may be combined with prior roadmap images to make rapid before-after comparisons and/or to track relative progress of the interventional device in the patient's body. Additionally, the low-latency imaging may also allow the surgeon to quickly jump to another region, for example, to verify if a current interventional procedure has resulted in injury and bleeding in surrounding tissues before continuing with the procedure at the site of intervention.

Furthermore, in one embodiment, the MRI system 100 may allow for automatic or interactive identification and labeling of scan planes, anatomical landmarks, and other features of interest. As previously noted, the identification and labeling of scan planes and anatomical landmarks in real-time during MR imaging is facilitated by the comprehensive anatomical awareness of the MRI system 100 gleaned from previously available information that may be continually updated based on recent MRI scans of the patient 112.

For example, in certain embodiments, the MRI system 100 allows the medical practitioner 152 to query the navigation system 101, for example, to identify a current region being imaged or a current position of the interventional device 166 based on a current position of a cursor on a GUI. Further, the operator may be allowed to request for identification and labeling of a surrounding region by indicating the region on the display 156. Additionally, the operator may also be allowed to request for diagnostic scans to assess a medical condition of the imaged region based on an analysis of image-derived parameters.

To that end, in certain embodiments, the navigation subsystem 101 may include an analysis unit 168 and a state machine 170 to service the operator requests. In one embodiment, for example, the analysis unit 168 may be configured to analyze images processed by the image processor 162, for example, for identifying landmarks, calculating scan planes, or quantifying tissue functions. Moreover, in certain embodiments, the analysis unit 168 may allow identification and labeling of the features of interest in offline mode and/or in real-time during the MRI scan.

As previously noted, the analysis unit 168 may perform the analysis based on knowledge derived from stored surgical atlases, anatomical models, reference images, expert systems, heuristic algorithms, Haar-like features, Scale Invariant Feature Transform (SIFT), and/or other standardized information. Specifically, the analysis unit 168 may evaluate and compare acquired images of the patient 112 with the standardized information to assess if structural and/or functional characteristics, such as size and shape of the imaged region differ substantially from the standardized information corresponding to that region. In one embodiment, the analysis unit 168 may automatically compare currently labeled features with those from a previous saved examination, for example, to estimate and highlight changes such as indicative of a pathological condition, such as proliferation of a tumor. Moreover, the analysis unit 168 may also account for variation in age, size, or sex of the patient 112 while making the assessments.

In certain embodiments, the navigation subsystem 101 allows the medical practitioner 152 to indicate what information is sought from the analysis unit 168. The analysis unit 168 may then be configured accordingly to analyze and communicate the labeled and identified features of interest, values of estimated structural and functional characteristics, results of the analysis, and any specific trends that were identified. In one embodiment, such information may be conveyed to the medical practitioner 152 as a report, visually on the display 156 through one or more colors, and/or audibly through the audio output device 160. Such audio-visual cues aid the medical practitioner 152 in reconfiguring appropriate imaging and display parameters in real-time based on changing imaging and/or device navigation requirements for further scans or interventions.

In certain embodiments, the MRI system 100 may also allow control over the reconfiguration of the imaging and display parameters, for example by providing feedback to the medical practitioner 152, to ensure that reconfigured parameters adhere to user-based and/or protocol-based mandates. To that end, the analysis unit 168 uses knowledge of a surgical interventional procedure as well as structural and functional characteristics of the target VOI being imaged for identifying features of interest and other useful information. The analysis unit 168 may derive this knowledge, as previously noted, from stored procedural information, surgical atlas, or models and/or self or assisted-learning systems. The analysis unit 168 may then use the derived knowledge to provide appropriate feedback for aiding in subsequent imaging and/or device guidance.

Further, the state machine 170 may determine the reconfiguration of the imaging and display parameters for the subsequent imaging based on the image analysis performed by the analysis unit 168. Particularly, the state machine 170 may be configured to track a current imaging state, transition into a next imaging state, or terminate scanning based on input received from the analysis unit 168. In certain embodiments, transitioning into the next imaging state, for example, may include selecting a next set of imaging parameters for a focused scan, furthering navigation and/or displaying associated patient information, while eliminating or reducing scans of little or no diagnostic value.

Accordingly, in one embodiment, the state machine 170 may be programmed, for example, using a decision tree or procedure stored in the storage repository 140, for determining imaging transitions. Alternatively, the state machine 170 may be programmed based on interactive input received from the medical practitioner 152 in real-time during imaging. The state machine 170, thus programmed, may allow reconfiguration of imaging and display parameters to direct scanning of a target VOI along a specific path based on a recently analyzed image or the received input. The state machine 170, for example, may direct a subsequent higher resolution scan towards a smaller FOV for classifying a lesion found in a previously analyzed image.

Alternatively, when performing a known medical procedure such as a transjugular intrahepatic portosystemic shunt (TIPS) procedure, the state machine 170 may configure the navigation subsystem 101 to direct scanning towards a large FOV that includes a target path connecting the portal vein of the patient 112 to one of the hepatic veins for stenting. Particularly, information from the state machine 170 may aid in configuration of the imaging and display parameters to allow generation of multi-plane or three-dimensional (3D) reference images that are indicative of a suitable path for the interventional device 166 to follow. Similarly, upon receiving a selection of a desired imaging task, the state machine 170 may aid the navigation subsystem 101 in determining a suitable scan plane for the desired imaging task based on the analyzed images. Specifically, the state machine 170 may assist the navigation subsystem 101 to determine the suitable scan plane based on a location of the ROI determined by the analysis unit 168, for example, with reference to a current scanner or patient position, scanner geometry, patient size, desired resolution, contrast, and/or gradient amplitude. Additionally, in certain embodiments, the state machine 170 may also determine an actual or natural path through the body for performing a specific procedure.

Further, the state machine 170 may communicate the determined information such as the path or scan plane to the navigation subsystem 101. The navigation system 101, in turn, may provide audio-visual guidance to the medical practitioner 152 to achieve the desired scan plane, position the scanner or the patient, and/or navigate the interventional device 166 through the patient vasculature. For example, the navigation subsystem 101 may display the determined path on the multi-planar images as a guide for the medical practitioner 152, while also providing real-time interventional device tracking.

Accordingly, in one embodiment, the navigation subsystem 101 may be configured to display the reconstructed, and optionally standard or previously available images on the display 156. Particularly, a manner of the display of images may be so as to allow the medical practitioner 152 to manipulate the interventional device 166 such that an icon representing the interventional device 166 coincides with the displayed path on the images as the interventional device 166 advances towards a target position. In certain embodiments, continual image processing by the analysis unit 168 and the state machine 170 may also allow the navigation subsystem 101 to provide a warning, for example through the display 156 and/or the audio output device 160, if the interventional device 166 deviates substantially from the target path.

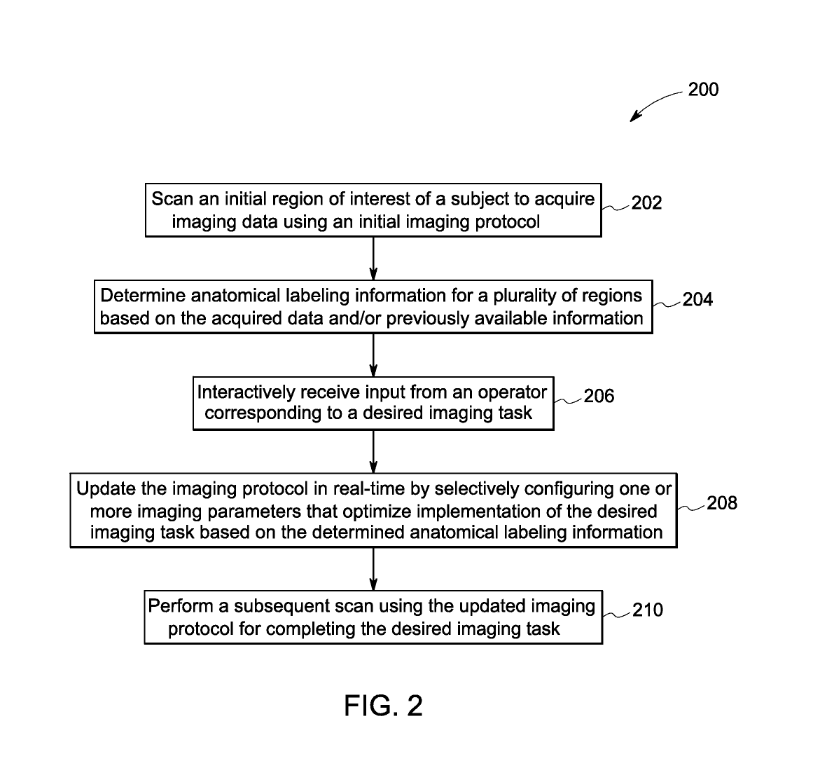

Embodiments of the present disclosure, thus, allow for greater flexibility and consistent performance in imaging the patient 112. Particularly, the interactive reconfiguration of the imaging parameters using anatomical knowledge derived from previous scans or stored information allow optimization of subsequent scans to achieve the desired imaging task. Such optimization, in turn may allow for a reduction in overall scanning time and increase in patient throughput irrespective of expertise or skill of the medical practitioner 152. An embodiment of such an interactive navigation and MR imaging method for optimizing desired imaging tasks will be described in greater detail with reference to FIG. 2.

FIG. 2 illustrates a flow chart 200 depicting an exemplary method for interactive body navigation and MR imaging. Embodiments of the exemplary method may be described in a general context of computer executable instructions on a computing system or a processor. Generally, computer executable instructions may include routines, programs, objects, components, data structures, procedures, modules, functions, and the like that perform particular functions or implement particular abstract data types.

Embodiments of the exemplary method may also be practised in a distributed computing environment where optimization functions are performed by remote processing devices that are linked through a wired and/or wireless communication network. In the distributed computing environment, the computer executable instructions may be located in both local and remote computer storage media, including memory storage devices.

Further, in FIG. 2, the exemplary method is illustrated as a collection of blocks in a logical flow chart, which represents operations that may be implemented in hardware, software, or combinations thereof. The various operations are depicted in the blocks to illustrate the functions that are performed, for example, during imaging data acquisition, processing, and imaging protocol update phases of the exemplary method. In the context of software, the blocks represent computer instructions that, when executed by one or more processing subsystems, perform the recited operations.

The order in which the exemplary method is described is not intended to be construed as a limitation, and any number of the described blocks may be combined in any order to implement the exemplary method disclosed herein, or an equivalent alternative method. Additionally, certain blocks may be deleted from the exemplary method or augmented by additional blocks with added functionality without departing from the spirit and scope of the subject matter described herein. For discussion purposes, the exemplary method will be described with reference to the elements of FIG. 1.

As previously noted, availability of high-quality images indicative of a current structural, functional and/or pathological condition of a target VOI and regions surrounding the target VOI of a patient is particularly advantageous in medical diagnosis, surgical and interventional procedures. Many medical procedures are increasingly employing MRI for enhanced characterization of soft tissues such as bone marrow, blood flow, and tissue contrasts without use of ionizing radiation typically used by X-ray and CT systems. Particularly, an MRI system, such as the MRI system 100 of FIG. 1, generates images having high spatial resolution for investigating minute features within a patient. Accurate characterization of specific features, for example, corresponding to the thoracic cavity allows for a better understanding of the physiology of heart and lungs, which in turn, aids in early detection of various cardiovascular and lung diseases. Additionally, the MRI system may include a navigation subsystem, such as the navigation subsystem 101 of FIG. 1, for accurate localization and navigation of an interventional device such as the interventional device 166 of FIG. 1 within the vascular system.

Accordingly, embodiments of the present method describe techniques for interactive and real-time navigation and MR imaging. Particularly, embodiments of the present disclosure allow for automated customization of imaging parameters that optimize various imaging tasks selectively input by the operator in real-time during imaging. To that end, in one embodiment, a patient is suitably positioned on an examination table associated with the MRI system for imaging, device tracking, and/or providing therapy to a target VOI, for example, a region proximal the patient's liver. Particularly, the patient may be positioned such that the target VOI is positioned within a FOV that is suitable for a selected imaging task, such as for diagnostic imaging, or for providing intervention.

To that end, at step 202, an initial volume of interest (VOI) of a subject is scanned using an initial imaging protocol to acquire imaging data. In one embodiment, the initial imaging protocol may be selected based on stored instructions that determine an appropriate protocol for use with the desired imaging task. In another embodiment, the initial imaging protocol may be determined based on a physical and/or a medical condition of the patient. Alternatively, the initial imaging protocol may be determined based on operator input received by the MRI system or the navigation subsystem. As used herein, the term “initial imaging protocol” may be used to refer to one or more specific combinations of imaging parameters such as FOV, scan plane, gradient amplitude, scan sequence, and timing, and so on, that allow acquisition of imaging data having desired characteristics, such as spatial resolution and/or quality from the initial VOI.

Further, at step 204, the acquired data is processed for identifying one or more features of interest corresponding to the initial VOI. To that end, in one embodiment, the processed data may be used to generate one or more images corresponding to the initial VOI. Further, these images may be compared with one or more of surgical atlases, anatomical models, reference images, and one or more prior examination images for identifying the features of interest and/or one or more anatomical landmarks. Alternatively, the generated images may be segmented, for example using level sets, region growing, graph cuts, SIFT, and/or fuzzy clustering techniques, for identifying the features of interest and/or the anatomical landmarks. In certain embodiments, keypoint detection techniques such as Haar filters or local binary patterns may also be used with and/or without a cascade for segmenting the images for identifying desired features of interest.

Accordingly, specific segmentation planes may be selected based on the specific information sought for diagnosing, device tracking, and/or assessing interventional and/or therapeutic progress. The images, thus segmented, may be used for identifying features of interest in the ROI and estimating structural parameters that may be indicative of the physiology of the ROI, thus aiding in early detection of various diseases. In certain other embodiments, however, the features of interest may be identified using algorithmic processing, for example, through use of various feature-detection algorithms. The identified features of interest may then be used by the navigation subsystem to provide navigational guidance for setting up and/or performing subsequent scans.

To that end, in certain embodiments, the navigation subsystem may label the identified features to provide visual and/or audio indications to an operator for performing one or more desired imaging tasks. For example, the identified features of interest may be indicative of a specific view of a region in the patient' body, and thus, may be highlighted by the navigation subsystem to allow the operator to determine coordinates of a desired scan plane for a subsequent scan. Alternatively, the identified features may correspond to an interventional device, such a catheter coupled to a reflective marker, which may be highlighted in real-time on a display. Such highlighting may allow the operator to select parameters that allow optimal implementation of a desired imaging task.

Accordingly, at step 206, the MRI system may be configured to receive input from the operator corresponding to the desired imaging task. For example, the operator may select a specific region for a subsequent scan or track progress of the interventional device during the course of the intervention through a selectable interface such as a control panel or a GUI on an associated display. Alternatively, the MRI system may be configured to receive verbal instructions from the operator that may be processed into operational instructions for implementing the desired task in real time. By way of example, the operator may request the navigation subsystem in the MRI system to identify a current region being imaged, for example, as indicated by a current cursor position on a GUI on the display or by orally asking, “where am I?” The navigation subsystem may also receive and implement requests for providing desired views, for example, an axial view of a current region.

Further, the operator may request the navigation subsystem to scan another ROI located at a specified relative distance from the initial VOI, for example, providing instructions for scanning 10 millimeters (mm) anterior to a current position, scanning 5 mm left of current FOV and scanning two slices posterior of a surgical device. Particularly, in one embodiment, the navigation subsystem may also allow representation of a current slice as a semi-transparent plane to allow visualization of underlying anatomy for better surgical guidance.

In certain embodiments, the navigation subsystem may also allow scanning of regions that may be positioned between two or more identified features of interest in the initial VOI, or in-plane with two or more features of interest, orally or by indicating the regions on the display. For example, an initial MRI scan of a thoracic region of the patient may result in identification and/or labeling of the patient's heart. In such a scenario, the navigation subsystem may allow the operator to request imaging of the heart with user-specified valves in the scan plane. Similarly, in certain embodiments, the operator may orally, or through selectable inputs request such as imaging from the heart to the liver.

Further, in response to the received inputs, at step 208, the imaging protocol may be updated in real-time. Specifically, the imaging protocol may be updated to optimize implementation of the desired imaging task by selectively configuring one or more imaging parameters using the processed imaging data. In one embodiment, for example, updating the imaging protocol may include selecting and/or configuring one or more of an imaging plane, scanning trajectory, patient position, or gradient amplitude suitable for imaging the selected ROI. In certain embodiments, updating the imaging protocol may include selectively configuring spatial resolution, temporal resolution, and/or image contrast as desired for one or more subsequent scans. In certain further embodiments, updating the imaging protocol may include determining a suitable scan sequence, image reconstruction technique and/or a type, frequency and/or timing of audio-visual output such as display rendering and color highlighting based on the feedback sought from the navigation subsystem during the medical procedure.

By way of example, in certain embodiments, the navigation subsystem is configured to scan a selected region of the patient's body using a known scan plane, for example, for performing a cardiac short-axis scan. In these embodiments, the navigation subsystem may use stored information corresponding to scanner geometry and patient anatomy to automatically and/or interactively advance a patient table to an appropriate location so as to position the correct anatomy within the scanner's FOV. The navigation subsystem may employ the ability to scan a known body part to momentarily jump to a different part of the patient anatomy, scan the part, and then return to the previous anatomy for further scanning. By way of example, a surgeon may use this ability during surgery to jump to a surrounding region and obtain corresponding scans to ascertain if any bleeding has occurred in the surrounding region.

Generally, when using a conventional MRI system, determination of a medical condition or identification of an accurate scan plane is based on the surgeon's experience and skill. In contrast to the conventional MRI systems, embodiments of the present navigation subsystem aid the operator in identifying and selectively configuring the imaging parameters that optimize the implementation of the desired imaging task. For example, the MRI system may be configured to provide audio and/or visual indications and instructions to the operator for selecting an appropriate imaging plane. The instructions may provide continual directions to the operator to move, for example, left, right, top, or bottom by a specified distance relative to a current position to achieve the desired imaging plane.

Further, at step 210, a subsequent scan may be performed using the updated imaging protocol for completing the desired imaging task, such as imaging a specified ROI of the patient at high resolution or communicating patient information to the surgeon for diagnosis and/or treatment planning. The method of MR imaging, such as described herein, allows for identification and highlighting of the features of interest in the FOV in real-time during imaging. The MRI system may further reconstruct and render the images on the display in real-time as the interventional device progresses through the patient's body based on interactive operator input.

To that end, as previously noted, the navigation subsystem may actively track RF coils embedded in an interventional device at known to determine a position of the interventional device in relation to the surrounding tissues. Alternatively, the navigation subsystem may allow for co-registration of MR images with standard images corresponding to the FOV that are derived from an atlas, an anatomical model, and/or previous examinations. The co-registered images may then be used to allow the operator to assess the movement of the interventional device in relation to the surrounding tissues. In certain embodiments, the navigation subsystem may be configured to provide an audible and/or visual warning to the operator upon determining that the interventional device has deviated from a determined path of navigation. The navigation subsystem may further provide audio and/or visual feedback based on a current position of the interventional device to guide the operator to return to the determined path of navigation. Once the operator returns to the suggested navigational path, the navigation subsystem may be configured to terminate any warning or alarms issued.

Embodiments of the present systems and methods, thus, provide an anatomy-aware MRI system for optimizing various imaging tasks. As previously noted, the MRI system may acquire this anatomy awareness based on anatomical atlases, models, acquired imaging information, heuristic and other related algorithms and/or self-learning systems. The anatomy awareness may allow the operator to query the MRI system to label the anatomy under the cursor, return a current location of the cursor with a selectable level of detail or to provide a suggested imaging parameters such as a scan plane or a path for navigation of a surgical or an interventional device in real-time upon receiving an interactive input from the operator. As previously noted, the interactive input may be received by the MRI system via various means such as verbal instructions and/or selection on a GUI or a control panel. Further, embodiments of the system and method allow for iterative updates to the imaging protocol in real-time to determine imaging parameters that optimize the implementation of the imaging task indicated by the operator.

Particularly, the interactive reconfiguration of the imaging parameters using anatomical knowledge derived from previous scans or stored information allow real-time optimization of subsequent scans to achieve the desired imaging task. Such optimization, in turn may allow for a reduction in overall scanning time and increase in patient throughput irrespective of expertise or skill of the operator. Additionally, the MRI system may be configured to provide feedback and/or warning based on operator's actions, thus providing great flexibility, accuracy, and consistent quality in imaging the patient.

It may be noted that the foregoing examples, demonstrations, and process steps that may be performed by certain components of the present systems, for example, by the navigation subsystem 101, the system controller 104, the processing subsystem 132, and the dedicated image processor 162 may be implemented by suitable code on a processor-based system, such as a general-purpose or a special-purpose computer. It may also be noted that different implementations of the present disclosure may perform some or all of the steps described herein in different orders or substantially concurrently.

Additionally, the functions may be implemented in a variety of programming languages, including but not limited to Ruby, Hypertext Preprocessor (PHP), Perl, Delphi, Python, C, C++, or Java. Such code may be stored or adapted for storage on one or more tangible, machine-readable media, such as on data repository chips, local or remote hard disks, optical disks (that is, CDs or DVDs), solid-state drives, or other media, which may be accessed by the processor-based system to execute the stored code.

Although specific features of various embodiments of the present disclosure may be shown in and/or described with respect to some drawings and not in others, this is for convenience only. It is to be understood that the described features, structures, and/or characteristics may be combined and/or used interchangeably in any suitable manner in the various embodiments, for example, to construct additional assemblies and techniques for use in interactive MRI.

While only certain features of the present disclosure have been illustrated and described herein, many modifications and changes will occur to those skilled in the art. It is, therefore, to be understood that the appended claims are intended to cover all such modifications and changes as fall within the true spirit of the invention.

1. A method for magnetic resonance imaging, comprising:
scanning an initial region of interest of a subject to acquire imaging data using an initial imaging protocol;
determining anatomical labeling information corresponding to a plurality of regions corresponding to the subject based on the acquired data, previously available information, or a combination thereof, wherein determining anatomical labeling information comprises identifying one or more features of interest corresponding to the initial region of interest;
interactively receiving input from an operator corresponding to a desired imaging task;
updating the imaging protocol in real-time by selectively configuring one or more imaging parameters that optimize implementation of the desired imaging task based on the determined anatomical labeling information; and
performing a subsequent scan using the updated imaging protocol for completing the desired imaging task.

2. The method of claim 1, wherein the initial imaging protocol is input by the operator.

3. The method of claim 1, wherein the initial imaging protocol is determined based on a medical procedure being undertaken.

4. The method of claim 1, further comprising providing automated navigation for performing the subsequent scan for completing the desired task.

5. The method of claim 4, wherein identifying the features of interest comprises:
generating one or more images using the processed data;
segmenting the images for identifying the features of interest, one or more anatomical landmarks, or a combination thereof;
estimating one or more structural parameters corresponding to the identified features of interest, the anatomical landmarks, or a combination thereof, based on the segmented images.

6. The method of claim 4, wherein identifying the features of interest comprises:
generating one or more images using the processed data; and
identifying the features of interest, one or more anatomical landmarks, or a combination thereof, based on heuristics, a feature detection algorithm, a comparison of the images with one or more of a surgical atlas, an anatomical model and one or more prior examination images, or combinations thereof.

7. The method of claim 4, wherein identifying the features of interest comprises labeling the identified features of interest, one or more anatomical landmarks determined from the processed data, or a combination thereof, on an associated display.

8. The method of claim 4, wherein identifying the features of interest comprises providing an audio indication corresponding to the identified features of interest, one or more anatomical landmarks determined from the processed data, or a combination thereof.

9. The method of claim 4, wherein identifying the features of interest comprises highlighting one or more of the identified features of interest and one or more anatomical landmarks determined from the processed data in real-time on an associated display during imaging.

10. The method of claim 4, wherein the desired task comprises determining and highlighting a path of navigation for an interventional device based on one or more of the identified features of interest and one or more anatomical landmarks determined from the anatomical labeling information in real-time on an associated display.

11. The method of claim 10, further comprising tracking progress of the interventional device along the path of navigation.

12. The method of claim 11, further comprising providing a warning if the interventional device deviates from the path of navigation.

13. The method of claim 12, further comprising terminating the warning once the interventional device returns to the path of navigation.

14. The method of claim 4, further comprising providing audio feedback, visual feedback, or a combination thereof, to the operator for selectively configuring the imaging parameters that optimize implementation of the imaging task.

15. The method of claim 4, further comprising receiving a selection of a desired imaging task from the operator in real-time during the magnetic resonance imaging.

16. The method of claim 15, wherein the imaging task comprises scanning another region of interest using a further operator-specified scan plane relative to an initial scan plane used to scan the initial region of interest.

17. The method of claim 15, wherein the imaging task comprises scanning another region of interest positioned between two or more identified features of interest, or in-plane with two or more identified features of interest.

18. The method of claim 4, wherein identifying the one or more features of interest corresponding to the initial region of interest comprises identifying an organ of interest and receiving a request for imaging a desired view of the organ of interest.

19. The method of claim 4, wherein interactively receiving input from the operator corresponding to the desired imaging task comprises receiving audible instructions.

20. The method of claim 1, wherein updating the imaging protocol in real-time comprises selectively configuring one or more of an imaging plane, scanning trajectory, gradient amplitude, spatial resolution, temporal resolution, image contrast, scan sequence, image reconstruction technique, color highlighting and patient position that optimize implementation of the desired imaging task.

21. A magnetic resonance imaging system, comprising:
a scanner configured to scan an initial region of interest of a subject to acquire imaging data using an initial imaging protocol;
one or more input-output devices configured to interactively receive input from an operator corresponding to a desired imaging task; and
a processing subsystem operationally coupled to one or more of the scanner and the input-output devices, wherein the processing subsystem is configured to:
determine anatomical labeling information corresponding to a plurality of regions corresponding to the subject based on the acquired data, previously available information, or a combination thereof, wherein determining anatomical labeling information comprises identifying one or more features of interest corresponding to the initial region of interest; and
update the imaging protocol in real-time by selectively configuring one or more imaging parameters that optimize implementation of the desired imaging task based on the determined anatomical labeling information; and
a navigation subsystem operationally coupled to one or more of the processing subsystem and the input-output devices and configured to direct a subsequent scan using the updated imaging protocol for completing the desired imaging task.

22. The system of claim 21, wherein the one or more input-output devices comprise a microphone configured to receive the initial imaging protocol input by the operator.

23. The system of claim 21, wherein the one or more input-output devices comprise a display device comprising a graphical user interface for selectively configuring the one or more imaging parameters that optimize implementation of the desired imaging task.

24. A non-transitory computer readable medium that stores instructions executable by one or more processors to perform a method for interactive magnetic resonance imaging, comprising:
scanning an initial region of interest of a subject to acquire imaging data using an initial imaging protocol;
determining anatomical labeling information corresponding to a plurality of regions corresponding to the subject based on the acquired data, previously available information, or a combination thereof, wherein determining anatomical labeling information comprises identifying one or more features of interest corresponding to the initial region of interest;
interactively receiving input from an operator corresponding to a desired imaging task;
updating the imaging protocol in real-time by selectively configuring one or more imaging parameters that optimize implementation of the desired imaging task based on the determined anatomical labeling information; and
performing a subsequent scan using the updated imaging protocol for completing the desired imaging task.
